(19) United States
(12) Patent Application Publication
(10) Pub. No.: US 2011/0054228 A1
(43) Pub. Date: Mar. 3, 2011

(54) BLUE ELECTROLUMINESCENT COMPOUNDS WITH HIGH EFFICIENCY AND DISPLAY DEVICE USING THE SAME

(75) Inventors: Sung-Min Kim, Seoul (KR); Bong Ok Kim, Seoul (KR); Mi Young Kwak, Seoul (KR); Hyuck Joo Kwon, Seoul (KR); Young Jun Cho, Seoul (KR)

(73) Assignee: GRACEL DISPLAY INC., SEOUL (KR)

(21) Appl. No.: 12/743,188

(22) PCT Filed: Nov. 22, 2007
(86) PCT No.: PCT/KR07/05912

§ 371 Date: Nov. 8, 2010

(51) Int. Cl. C07C 13/62

(52) U.S. Cl. 585/26

(57) ABSTRACT

The present invention relates to novel organic electroluminescent compounds and display devices comprising the same. The organic electroluminescent compounds according to the present invention exhibit high luminous efficiency and excellent life property, so that an OLED device having very good operation life can be prepared therefrom.

### TECHNICAL FIELD

The present invention relates to novel organic electroluminescent compounds and display devices using the same as electroluminescent material.

### BACKGROUND ART

Three electroluminescent materials (for red, green and blue) are employed to realize a full-colored OLED display. The important issue is to develop red, green and blue electroluminescent materials with high efficiency and long life in order to enhance the overall character of the organic electroluminescent (EL) materials. From the aspect of function, the EL materials are divided into host materials and dopant materials. It is generally known that a device structure having the most excellent EL properties can be fabricated with an EL layer prepared by doping a dopant on a host. Recently, development of an organic EL device with high efficiency and long life comes to the fore as an urgent subject, and particularly urgent is development of a material with far better EL properties as compared to conventional EL materials as considering EL properties required for a medium to large size OLED panel. From this point of view, development of a host material is one of the most important issues to be settled. The desired properties for the solid state solvent and the host material (serving as an energy conveyer) are high purity and appropriate molecular weight to enable vacuum-deposition. In addition, glass transition temperature and thermal decomposition temperature should be high to ensure thermal stability. Further, the host material should have high electrochemical stability for providing long life. It is to be easy to form an amorphous thin layer, with high adhesiveness to other adjacent materials but without interlayer migration.

Conventional host materials include diphenylvinyl-biphenyl (DPVBi) from Idemitsu-Kosan and dinaphthyl-anthracene (DNA) from Kodak, but still requiring a number of improvements in terms of efficiency, life and color purity.

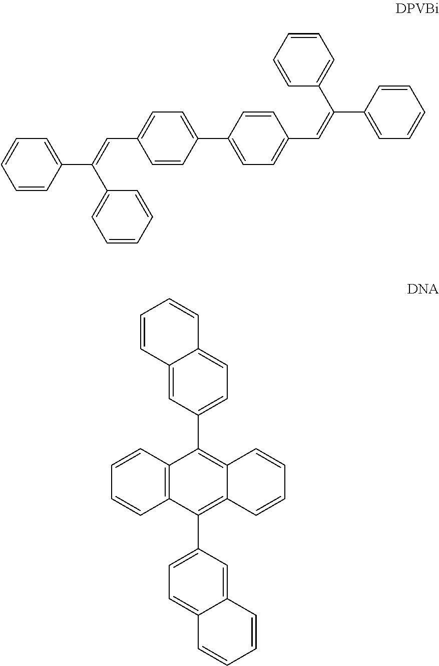

On order to develop a host material of high efficiency and long life, EL compounds based on different skeletals have been disclosed, such as dispiro-fluorene-anthracene (TBSA), ter-spirofluorene (TSF) and bitriphenylene (BTP). These compounds, however, did not result in color purity and luminous efficiency at a sufficient level.

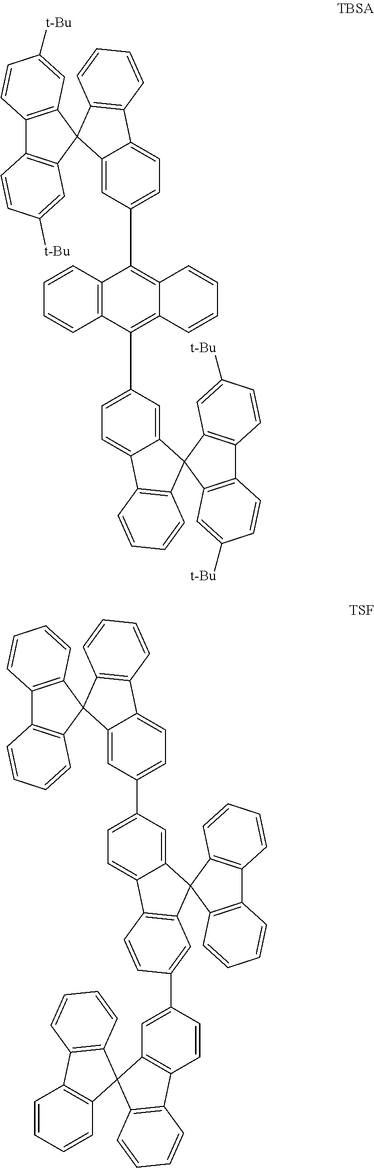

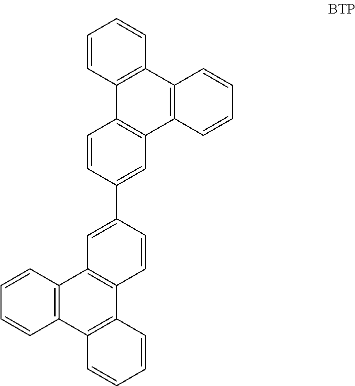

The compound TBSA as reported by Gyeongsang National University and Samsung SDI (Kwon, S. K. et al., Advanced Materials, 2001, 13, 1690; Japanese Patent Laid-Open JP 2002121547) showed luminous efficiency of 7.7 V at 3 cd/A, and relatively good color coordinate of (0.15, 0.11), but it is inappropriate for practical use. The compound TSF reported by Taiwan National University (Wu, C.-C. et al., Advanced Materials, 2004, 16, 61; US Patent Publication US 2005040392) showed relatively good external quantum efficiency of 5.3%, but it is still insufficient for practical use. The compound BTP reported by Chingwha National University of Taiwan (Cheng, C.-H. et al., Advanced Materials, 2002, 14, 1409; US Patent Publication 2004076852) showed luminous efficiency of 2.76 cd/A and relatively good color coordinate of (0.16, 0.14), but this is still insufficient for practical use.

### DISCLOSURE

### Technical Problem

The object of the present invention is to provide organic EL compounds having peculiar skeletal, which shows higher luminous efficiency as compared to conventional host materials, and appropriate color coordinate. Another object of the present invention is to provide display devices comprising the organic EL compounds.

### Technical Solution

The present invention relates to organic EL compounds represented by Chemical Formula (1), and display devices comprising the same.

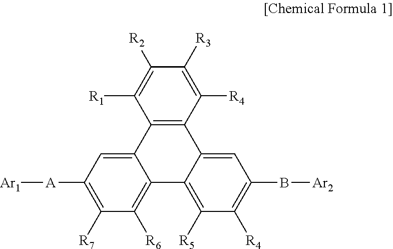

In the Chemical Formula, A and B independently represent a chemical bond or C₆-C₃₀ arylene, R₁ through R₇ independently represent hydrogen, a C₁-C₂₀ linear or branched alkyl group, or a C₆-C₃₀ aryl group, or R₁ through R₇ may form a fused ring by an alkylene linkage with an adjacent group selected from R₁ to R₇; Ar₁ and Ar₂ independently represent hydrogen, phenyl, naphthyl, anthryl or fluorenyl, wherein the phenyl, naphthyl, anthryl or fluorenyl may have one or more substituent(s) selected from the group consisting of C₁-C₂₀ linear or branched alkyl or alkoxy groups, C₆-C₃₀ aryl or heteroaryl groups and halogen; provided that Ar₁ and Ar₂ are not hydrogen at the same time; and

the arylene, aryl, heteroaryl, alkyl and alkoxy groups may be further substituted by C₁-C₂₀ linear or branched alkyl, aryl or halogen.

A and B independently represent a chemical bond or a group represented by one of the following chemical formulas.

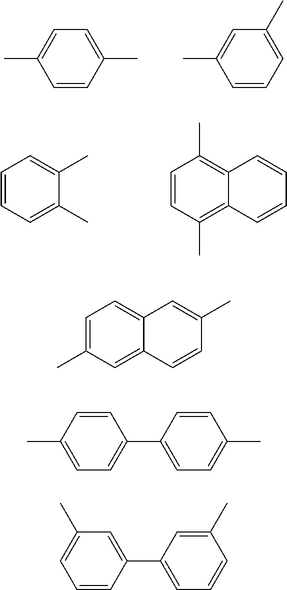

If A or B in the chemical formulas of the present invention does not contain an element but simply represents a linkage to Ar₁ or Ar₂, it is referred to as “a chemical bond”.

Group Ar₁ and Ar₂ independently represent an anthryl group represented by one of the following chemical formulas:

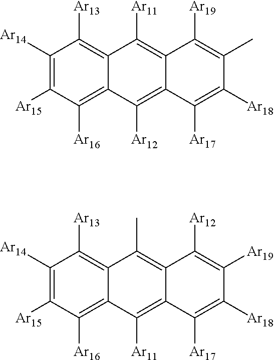

In the formulas, Ar₁ₗ through Ar₁₉ independently represent hydrogen, C₁-C₂₀ linear or branched alkyl or alkoxy group, C₆-C₃₀ aryl or heteroaryl group or halogen; and the aryl, heteroaryl, alkyl or alkoxy group may be further substituted by C₁-C₂₀ linear or branched alkyl, aryl or halogen.

In the compounds represented by Chemical Formula (1), R₁ through R₇ may be independently selected from the group consisting of hydrogen, methyl, ethyl, propyl, isopropyl, butyl, isobutyl, pentyl, hexyl, ethylhexyl, heptyl, octyl, isooctyl, nonyl, decyl, dodecyl, hexadecyl, phenyl, tolyl, biphenyl, benzyl, naphthyl, anthryl and fluorenyl.

The compounds represented by Chemical Formula (1) according to the present invention include the compounds represented by one of Chemical Formulas (2) to (5):

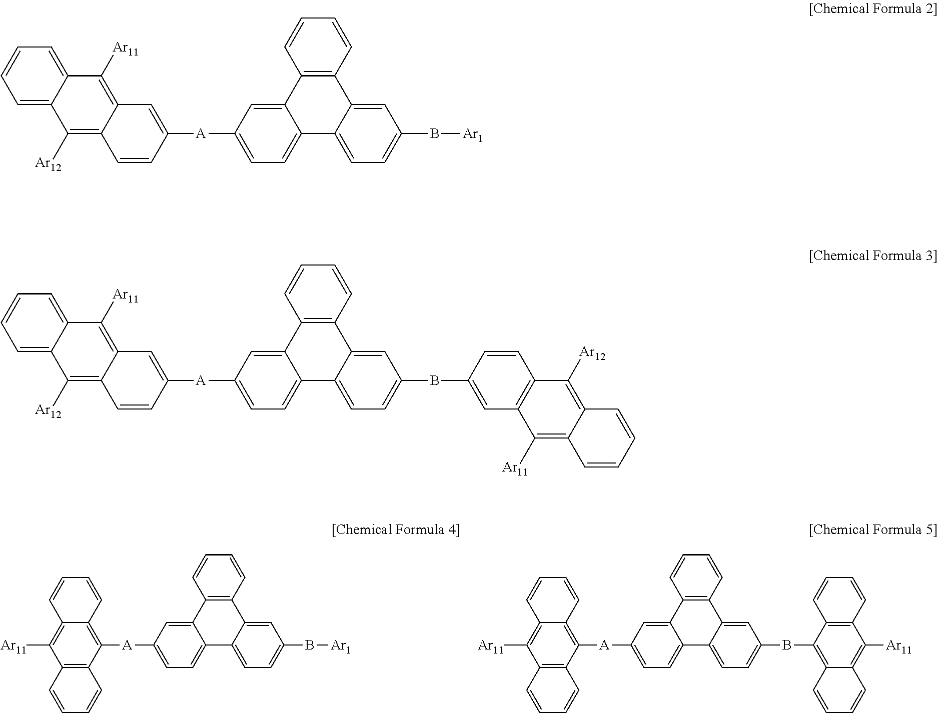

In Chemical Formulas (2) to (5), A and B are defined as in Chemical Formula (1), and Ar₁, Ar₁ₗ and Ar₁₂ are independently selected from the group consisting of phenyl, 4-tolyl, 3-tolyl, 2-tolyl, 2-biphenyl, 3-biphenyl, 4-biphenyl, (3,5-diphenyl)phenyl, 9,9-dimethyl-fluoren-2-yl, 9,9-diphenyl-fluoren-2-yl, (9,9-(4-methylphenyl)-fluoren)-2-yl, 1-naphthyl, 2-naphthyl, 1-anthryl, 2-anthryl, 3-anthryl and 2-spirofluorenyl.

The organic EL compounds represented by one of Chemical Formulas (2) to (5) are specifically exemplified by following compounds:

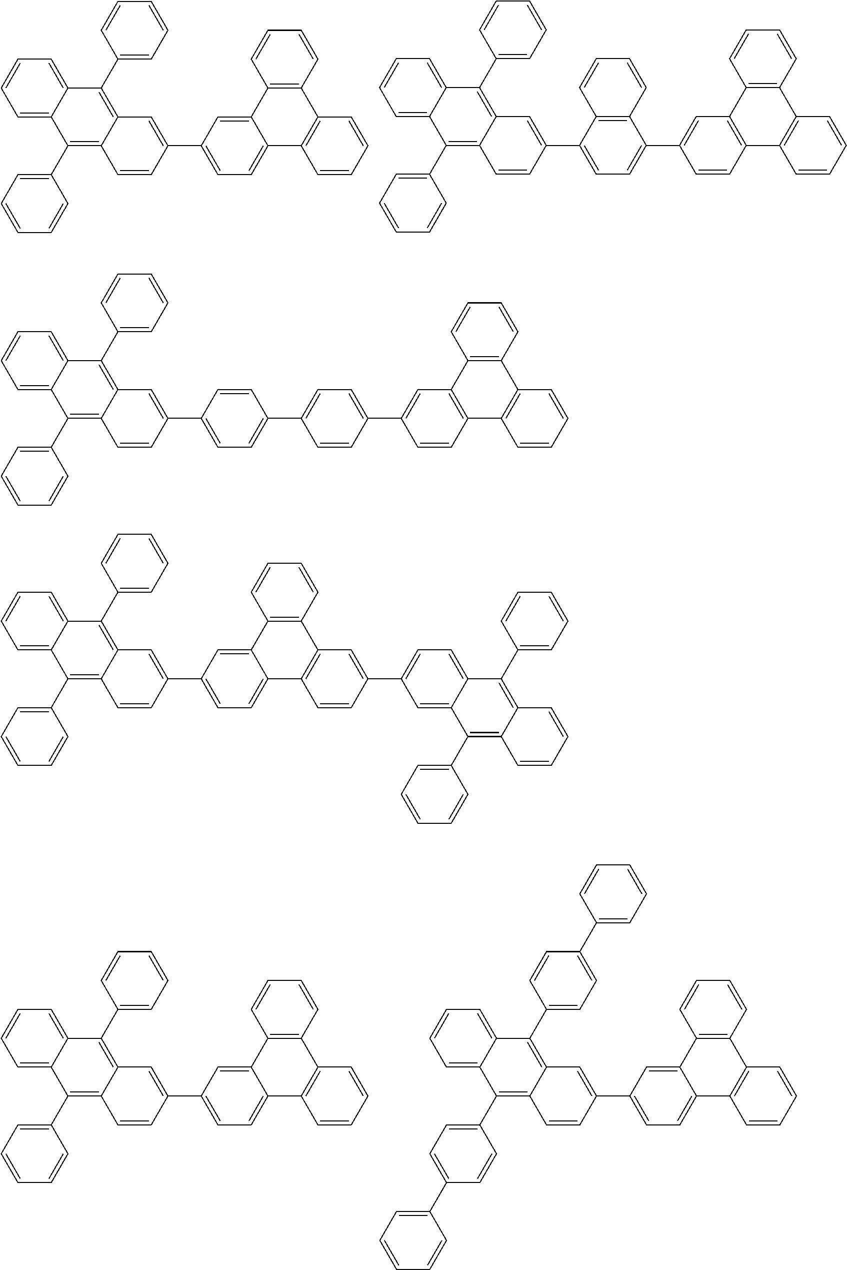

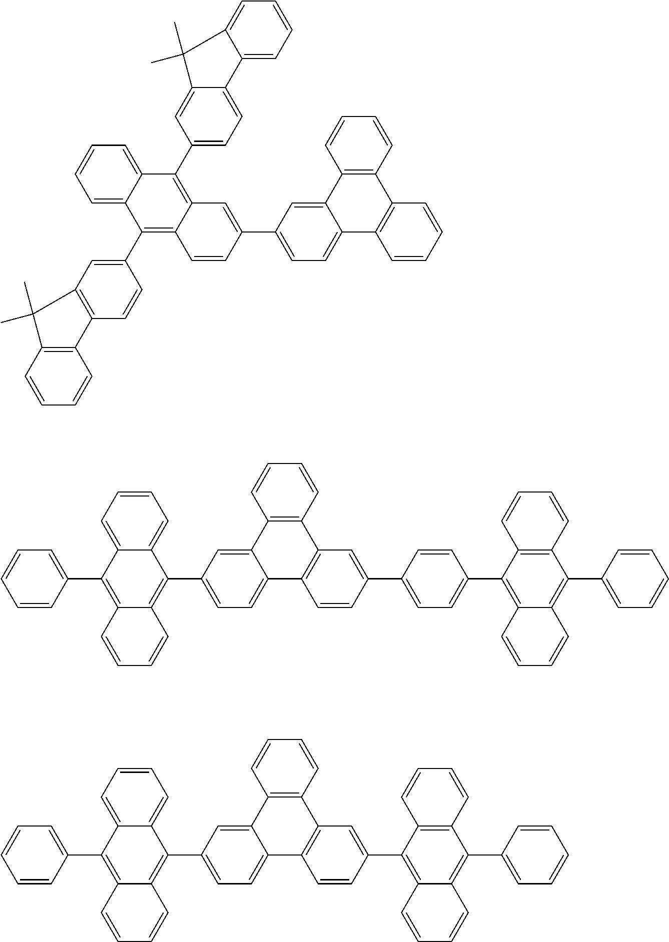

Other and further objects, features and advantages of the invention will appear more fully from the following description.

### BEST MODE

### Preparation Example 1

### Preparation of Compound (116)

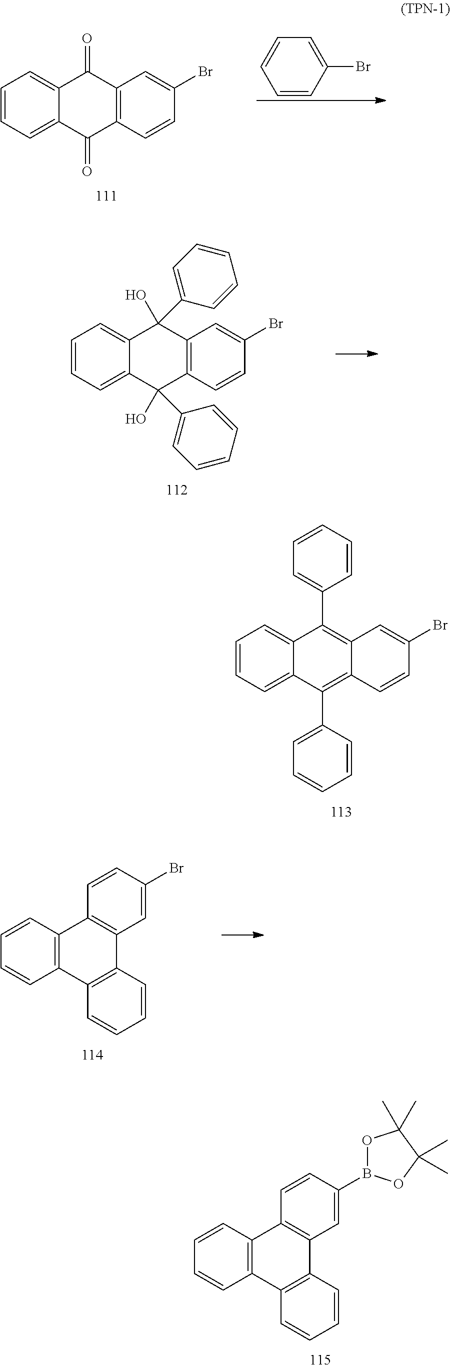

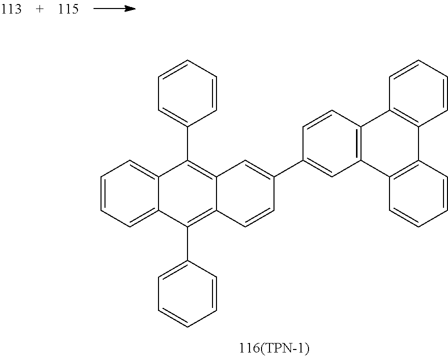

### Preparation of Compound (112)

Under nitrogen atmosphere, 1-bromobenzene (7.1 g, 45.0 mmol) was dissolved in tetrahydrofuran (100 mL), and n-BuLi (2.5 M in n-hexane) (27.0 mL, 67.5 mmol) was added dropwise at −78° C. thereto. After stirring for 2 hours, the reaction mixture was slowly added dropwise at −78° C. to a solution of 2-bromoanthraquinone (Compound III) (4.3 g, 15.0 mmol) dissolved in tetrahydrofuran (50 mL) at 25° C. under nitrogen atmosphere. The temperature was slowly raised from −78° C. to 25° C., and the reaction mixture was stirred for 12 hours. After quenching the reaction by adding saturated aqueous ammonium chloride solution (50 mL), the reaction mixture was extracted with ethyl acetate (100 mL), dried over anhydrous magnesium sulfate. Removal of the organic layer under reduced pressure and recrystallization from dichloromethane (100 mL) gave Compound (112) (5.7 g, 12.9 mmol).

### Preparation of Compound (113)

A reaction vessel was charged with Compound (112) (5.7 g, 12.9 mmol), potassium iodide (8.5 g, 51.4 mmol) and sodium hydrophosphate hydrate (NaHPO₂H₂O) (8.2 g, 77.2 mmol). Glacial acetic acid (50 mL) was added thereto in order to dissolve the content of the vessel, and the solution was stirred under reflux. After 18 hours of stirring, the reaction mixture was cooled to 25° C., and distilled water (400 mL) was added thereto. The solid produced was filtered and washed with excess amount of water. Washing with aqueous sodium hydroxide solution (100 mL) and recrystallization from n-hexane (300 mL) gave Compound (113) (4.2 g, 10.3 mmol).

### Preparation of Compound (115)

In tetrahydrofuran (100 mL), 1-bromotriphenylene (Compound 114) (5.0 g, 16.2 mmol) was dissolved, and n-BuLi (2.5 M in n-hexane) (9.7 mL, 24.3 mmol) was slowly added dropwise thereto at −78° C. After stirring for 1 hour, 2-isopropoxy-4,4,5,5-tetramethyl-1,3,2-dioxaborolane (9.0 g, 48.6 mmol) was added thereto at −78° C., and the resultant mixture was stirred at 25° C. for 2 hours. The reaction mixture was washed with water (100 mL), extracted with ethyl acetate (100 mL) and dried over anhydrous magnesium sulfate. Evaporation of the organic layer under reduced pressure and recrystallization from methanol (50 mL) gave solid, which was then filtered and dried to obtain Compound (115) (4.7 g, 10.0 mmol).

### Preparation of Compound (116)

A reaction vessel was charged with Compound (113) (4.2 g, 30.0 mmol) and the ester compound (Compound 115) (4.7 g, 10.0 mmol), and tetrakispalladiumtriphenylphosphine (Pd(PPh₃)₄) (1.2 g, 1.0 mmol) was added thereto. The mixture was dissolved in toluene (80 mL). To the solution, added were Aliquat 336 (0.5 g, 1.0 mmol) and aqueous 2M calcium carbonate solution (24 mL). The resultant mixture was stirred at 130° C. under reflux for 4 hours. To the precipitate formed, methanol (200 mL) was poured to form solid, which was then dissolved in chloroform (300 mL). After filtration, the organic layer was evaporated under reduced pressure. Recrystallization from tetrahydrofuran (30 mL) gave the target compound (116) (TPN-1) (2.2 g, overall yield: 38%).

¹H NMR (200 MHz, CDCl₃) δ=7.22-7.32 (m, 8H), 7.48-7.54 (m. 5H), 7.67-7.89 (t, 8H), 8.10-8.12 (m, 3H), 8.34 (d, 1H), 8.93-8.99 (m, 3H)

MS/FAB: 556.22 (found), 556.69 (calculated)

### Preparation Example 2

### Preparation of Compound (121)

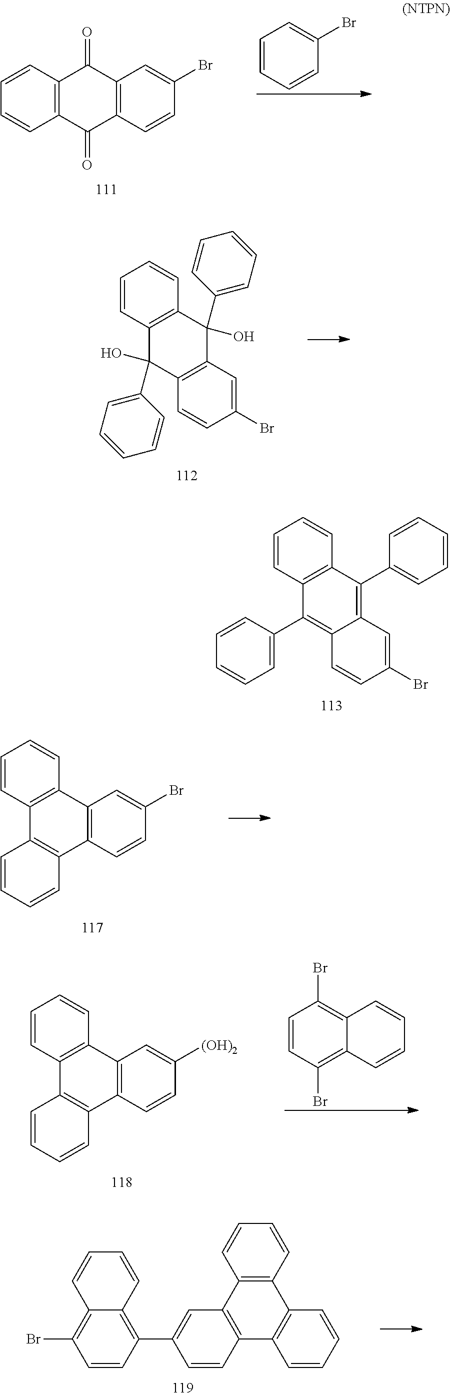

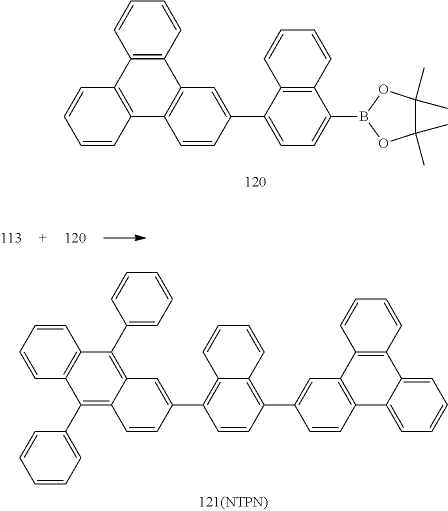

### Preparation of Compound (112)

Under nitrogen atmosphere, 1-bromobenzene (8.3 g, 53.1 mmol) was dissolved in tetrahydrofuran (100 mL), and n-BuLi (2.5 M in n-hexane) (29.5 mL, 73.7 mmol) was slowly added dropwise at −78° C. thereto. After stirring for 2 hours, the reaction mixture was slowly added dropwise at −78° C. to a solution of 2-bromoanthraquinone (Compound III) (5.0 g, 17.4 mmol) dissolved in tetrahydrofuran (100 mL) at 25° C. under nitrogen atmosphere. The temperature was slowly raised from −78° C. to 25° C., and the reaction mixture was stirred for 12 hours. After quenching the reaction by adding saturated aqueous ammonium chloride solution (500 mL), the reaction mixture was extracted with ethyl acetate (500 mL), dried over anhydrous magnesium sulfate. Evaporation of the organic layer under reduced pressure and recrystallization of the solid from dichloromethane (300 mL) gave Compound (112) (6.7 g, 15 mmol).

### Preparation of Compound (113)

A reaction vessel was charged with Compound (112) (6.7 g, mmol), potassium iodide (9.7 g, 60 mmol) and sodium hydropotassiumphosphate hydrate (NaHPO₂H₂O) (9.5 g, 90 mmol). Glacial acetic acid (50 mL) was added thereto to dissolve the content of the vessel, and the solution was stirred under reflux. After 18 hours of stirring, the reaction mixture was cooled to 25° C., and distilled water (100 mL) was added thereto. The solid produced was filtered and washed with excess amount of water. Washing with aqueous sodium hydroxide solution (200 mL) and recrystallization from n-hexane (200 mL) gave Compound (113) (5.0 g, 12.2 mmol).

### Preparation of Compound (118)

In tetrahydrofuran (280 mL), 1-bromotriphenylene (Compound 117) (10.6 g, 34.5 mmol) was dissolved under nitrogen atmosphere, and n-BuLi (2.5 M in n-hexane) (17.9 mL, 44.9 mmol) was slowly added dropwise thereto at −78° C. After stirring for 1 hour, trimethyl borate (7.2 g, 69 mmol) was added at low temperature, and the mixture was stirred while slowly raising the temperature to 25° C. After stirring for 16 hours, 10 M hydrochloric acid (30 mL) was added thereto. The resultant mixture was stirred for 1 hour, extracted with ethyl acetate (200 mL). The extract was washed with water (200 mL), dried over anhydrous magnesium sulfate, and the organic layer was evaporated under reduced pressure. The solid obtained was recrystallized from hexane (100 mL), and the filtered solid was dried under reduced pressure to obtain Compound (118) (9.0 g, 33.0 mmol).

### Preparation of Compound (119)

Compound (118) (9.2 g, 33.9 mmol), 1,4-dibromonaphthalene (8.8 g 30.8 mmol) and trans-dichlorobistriphenylphosphine palladium (II) (Pd(PPh₃)₂Cl₂ (2.1 g, 3.1 mmol) were dissolved in toluene (300 mL). After adding 2 M sodium carbonate solution (150 mL), the mixture was heated to 100° C., and reacted at the same temperature for 3 hours. The reaction mixture was extracted with dichloromethane (300 mL), and the extract was washed with aqueous sodium chloride solution (300 mL), dried over anhydrous magnesium sulfate, and filtered. After evaporating the organic layer under reduced pressure, the solid obtained was recrystallized from tetrahydrofuran (100 mL) to obtain the desired compound (119) (7.7 g, 17.7 mmol).

### Preparation of Compound (120)

Compound (119) (7.7 g, 17.7 mmol) was dissolved in tetrahydrofuran (200 mL) under nitrogen atmosphere, and n-BuLi (2.5 M in n-hexane) (10.6 mL, 26.6 mmol) was slowly added dropwise thereto at −78° C. After stirring the mixture for 1 hour, added was 2-isopropoxy-4,4,5,5-tetramethyl-1,3,2-dioxaborolane (6.6 g, 35.4 mmol), and the resultant mixture was stirred while raising the temperature to 25° C. The mixture was then extracted with ethyl acetate (300 mL), and the organic layer was washed with 300 mL of water, dried over anhydrous magnesium sulfate, and evaporated under reduced pressure. The solid obtained was recrystallized from methanol (150 mL), and the filtered solid was dried to obtain Compound (120) (7.6 g, 15.9 mmol).

### Preparation of Compound (121)

A reaction vessel was charged with Compound (113) (5.0 g, 12.2 mmol) and the ester compound (120) (7.6 g, 15.9 mmol), and tetrakispalladiumtriphenylphosphine (Pd(PPh₃)₄) (1.4 g, 1.2 mmol) was added thereto. After dissolving the mixture in toluene, added were Aliquat 336 (0.6 g, 1.2 mmol) and then aqueous 2 M calcium carbonate solution (30 mL). The reaction mixture was stirred at 130° C. under reflux for 4 hours. To the precipitate produced, poured was excess amount of methanol to form solid. The solid was dissolved in chloroform (300 mL), filtered, and the solvent was removed under reduced pressure. Recrystallization from tetrahydrofuran (300 mL) gave the target compound (121) (NTPN) (3.5 g, overall yield: 42%).

¹H NMR (200 MHz, CDCl₃) δ=7.22-7.32 (m, 10H), 7.48-7.67 (m. 12H), 8.10-8.12 (m, 3H), 8.34 (dd, 1H), 8.93-8.99 (m, 3H)

MS/FAB: 682.27 (found), 682.85 (calculated)

### Preparation Example 3

### Preparation of Compound (126)

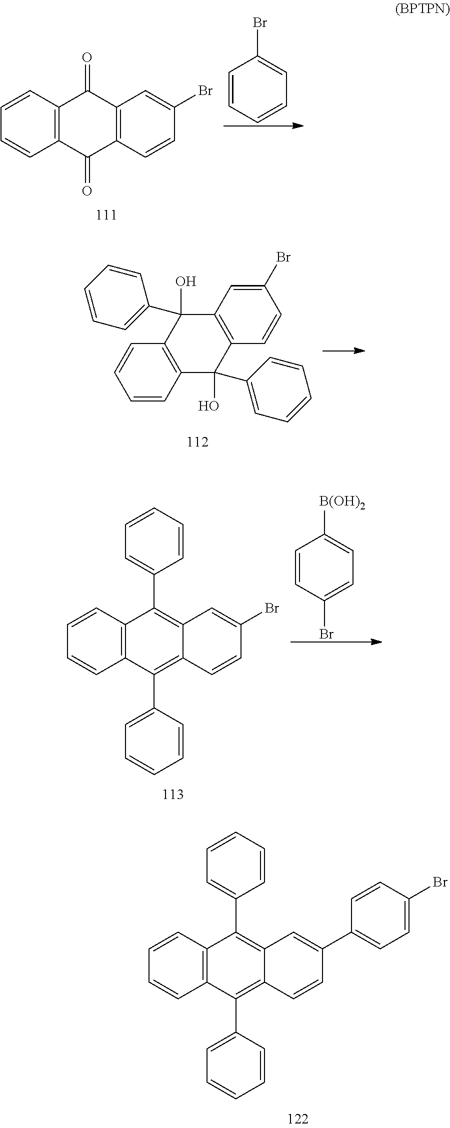

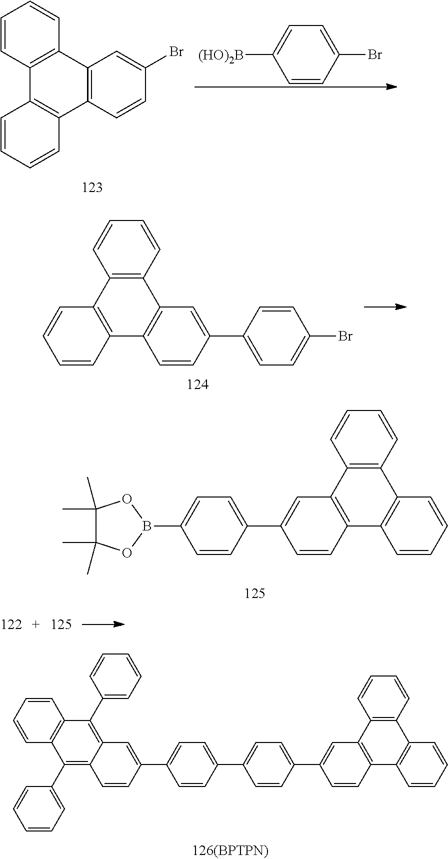

### Preparation of Compound (112)

Under nitrogen atmosphere, 1-bromobenzene (8.2 g, 52.2 mmol) was dissolved in tetrahydrofuran (150 mL), and n-BuLi (2.5 M in n-hexane) (31.3 mL, 78.3 mmol) was added dropwise at −78° C. thereto. After stirring for 2 hours, the reaction mixture was slowly added dropwise at −78° C. to a solution of 2-bromoanthraquinone (Compound III) (5 g, 17.4 mmol) dissolved in tetrahydrofuran (50 mL) at 25° C. under nitrogen atmosphere. The temperature was slowly raised from −78° C. to 25° C., and the reaction mixture was stirred for 12 hours. After quenching the reaction by adding saturated aqueous ammonium chloride solution (500 mL), the reaction mixture was extracted with ethyl acetate (500 mL), dried over anhydrous magnesium sulfate. Evaporation of the organic layer under reduced pressure and recrystallization from dichloromethane (300 mL) gave Compound (112) (6.6 g, 14 mmol).

### Preparation of Compound (113)

A reaction vessel was charged with Compound (112) (6.6 g, mmol), potassium iodide (9.3 g, 56 mmol) and sodium hydropotassiumphosphate hydrate (NaHPO₂H₂O) (8.9 g, 84 mmol). Glacial acetic acid (40 mL) was added thereto to dissolve the content of the vessel, and the solution was stirred under reflux. After 18 hours of stirring, the reaction mixture was cooled to 25° C., and distilled water (100 mL) was added thereto. The solid produced was filtered and washed with excess amount of water. Washing again with aqueous sodium hydroxide solution (200 mL) and recrystallization from n-hexane (200 mL) gave Compound (113) (5.3 g, 12.9 mmol).

### Preparation of Compound (122)

Compound (113) (4.9 g, 12 mmol), 4-bromophenylboronic acid (2.7 g, 13.2 mmol) and trans-dichlorobistriphenylphosphine palladium (II) (Pd(PPh₃)₂Cl₂ (0.9 g, 1.2 mmol) were dissolved in toluene (120 mL). After adding aqueous 2 M sodium carbonate solution (60 mL) with stirring, the mixture was heated at 120° C. under reflux and reacted for 3 hours. The reaction mixture was then extracted with dichloromethane (200 mL), washed with aqueous sodium chloride solution (200 mL), and dried over anhydrous magnesium sulfate. After evaporating the organic layer under reduced pressure, the solid obtained was recrystallized from tetrahydrofuran (100 mL) to obtain Compound (122) (5.0 g, 10.3 mmol).

### Preparation of Compound (124)

Compound (123), 1-bromotriphenylene (5.8 g, 19 mmol), 4-bromophenylboronic acid (4.2 g, 20.9 mmol) and trans-dichlorobistriphenylphosphine palladium (II) (Pd(PPh₃)₂Cl₂ (1.3 g, 1.9 mmol) were dissolved in toluene (200 mL). After adding aqueous 2 M sodium carbonate solution (100 mL) with stirring, the mixture was heated at 120° C. under reflux and reacted for 3 hours. The reaction mixture was then extracted with dichloromethane (200 mL), washed with aqueous sodium chloride solution (200 mL), dried over anhydrous magnesium sulfate, and filtered. After evaporating the organic layer under reduced pressure, the solid obtained was recrystallized from tetrahydrofuran (100 mL) to obtain Compound (124) (5.8 g, 15 mmol).

### Preparation of Compound (125)

Compound (124) (5.8 g, 15 mmol) was dissolved in tetrahydrofuran (150 mL) under nitrogen atmosphere, and n-BuLi (2.5 M in n-hexane) (9 mL, 22.5 mmol) was slowly added dropwise thereto at −78° C. After stirring the mixture for 1 hour, added was 2-isopropoxy-4,4,5,5-tetramethyl-1,3,2-dioxaborolane (5.6 g, 30 mmol), and the resultant mixture was stirred while raising the temperature to 25° C. The mixture was then extracted with ethyl acetate (300 mL), and the organic layer was washed with of water (300 mL), dried over anhydrous magnesium sulfate, and filtered. The organic layer was evaporated under reduced pressure, and the solid obtained was recrystallized from methanol (200 mL). Filtration and drying the solid gave Compound (125) (6.0 g, 13.9 mmol).

### Preparation of Compound (126)

A reaction vessel was charged with Compound (122) (5 g, 10.3 mmol) and the ester compound (125) (5.8 g, 13.4 mmol), and tetrakistriphenylphosphinepalladium (Pd(PPh₃)₄) (1.2 g, 1.0 mmol) was added thereto. After dissolving the mixture in toluene, added were Aliquat 336 (0.5 g, 1.03 mmol) and then aqueous 2 M calcium carbonate solution (30 mL). The reaction mixture was stirred at 120° C. under reflux for 6 hours. To the precipitate obtained, poured was excess amount of methanol (300 mL) to form solid. The solid was dissolved in chloroform (500 mL), filtered, and the solvent was removed under reduced pressure. Recrystallization from tetrahydrofuran (200 mL) gave the target compound (126) (BPTPN) (2.5 g, overall yield: 34%).

¹H NMR (200 MHz, CDCl₃) δ=7.22-7.32 (m, 8H), 7.48-7.54 (m. 13H), 7.67-7.73 (m, 3H), 7.82-7.89 (m, 5H), 8.03-8.05 (m, 1H), 8.10-8.18 (m, 3H), 8.93-8.95 (dd, 2H), 9.15 (dd, 1H)

MS/FAB: 708.28 (found), 708.89 (calculated)

### Preparation Example 4

### Preparation of Compound (129)

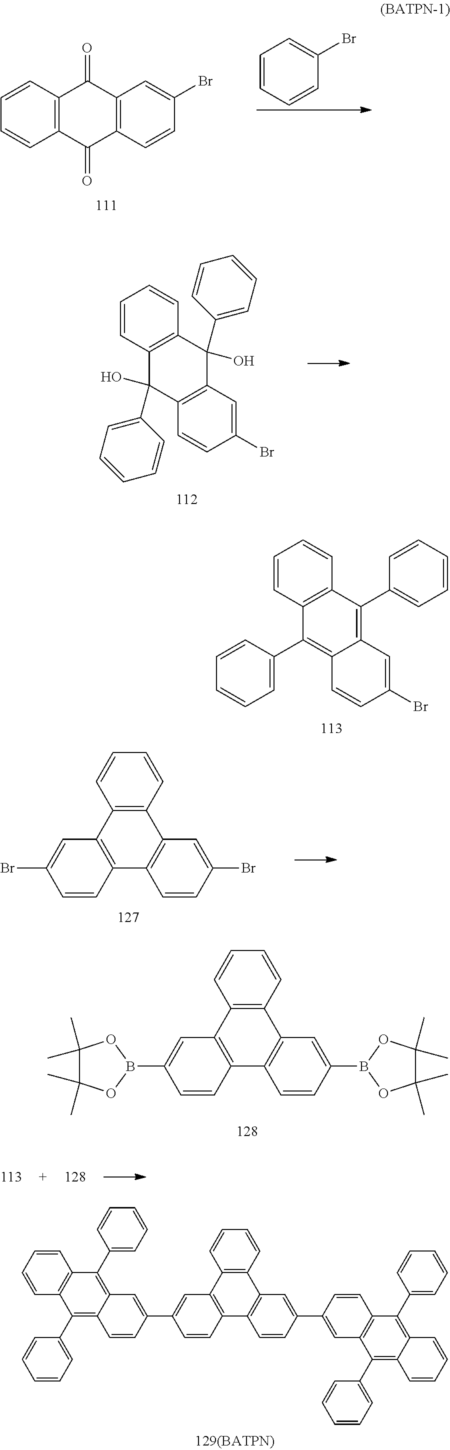

### Preparation of Compound (112)

Under nitrogen atmosphere, 1-bromobenzene (19.4 g, 123.6 mmol) was dissolved in tetrahydrofuran (250 mL), and n-BuLi (2.5 M in n-hexane) (74.16 mL, 185.4 mmol) was added dropwise at −78° C. thereto. After stirring for 2 hours, the reaction mixture was slowly added dropwise at −78° C. to a solution of 2-bromoanthraquinone (Compound III) (11.8 g, 41.2 mmol) dissolved in tetrahydrofuran (200 mL) at 25° C. under nitrogen atmosphere. The temperature was slowly raised from −78° C. to 25° C., and the reaction mixture was stirred for 12 hours. After quenching the reaction by adding saturated aqueous ammonium chloride solution (400 mL), the reaction mixture was extracted with ethyl acetate (400 mL), dried over anhydrous magnesium sulfate, and filtered. Evaporation of the organic layer under reduced pressure provided solid compound, which was then recrystallized from dichloromethane (200 mL) to obtain Compound (112) (15.5 g, 35 mmol).

### Preparation of Compound (113)

A reaction vessel was charged with Compound (112) (15.5 g, 35.0 mmol), potassium iodide (23.2 g, 140.0 mmol) and sodium hydropotassiumphosphate hydrate (NaHPO₂H₂O) (22.3 g, 210.0 mmol). Glacial acetic acid (90 mL) was added thereto to dissolve the content of the vessel, and the solution was stirred under reflux. After 18 hours of stirring, the reaction mixture was cooled to 25° C., and distilled water (200 mL) was added thereto. The solid produced was filtered and washed with excess amount (300 mL) of water. Washing again with aqueous sodium hydroxide solution (200 mL) and recrystallization from hexane (200 mL) gave Compound (113) (13.0 g, 31.8 mmol).

### Preparation of Compound (128)

In tetrahydrofuran (120 mL), 1,8-dibromotriphenylene (Compound 127) (4.6 g, 12.0 mmol) was dissolved, and n-BuLi (2.5 M in n-hexane) (14.4 mL, 35.9 mmol) was slowly added dropwise thereto at −78° C. After stirring for 1 hour, 2-isopropoxy-4,4,5,5-tetramethyl-1,3,2-dioxaborolane (23.4 g, 47.9 mmol) was added at low temperature, and the resultant mixture was stirred while slowly raising the temperature to 25° C. The reaction mixture was washed with water (300 mL), extracted with ethyl acetate (300 mL) and dried over anhydrous magnesium sulfate. Evaporation of the organic layer under reduced pressure and recrystallization from methanol (150 mL) gave solid, which was then filtered to obtain Compound (128) (5.0 g, 10.4 mmol).

### Preparation of Compound (129)

A reaction vessel was charged with Compound (113) (12.8 g, 33.0 mmol) and the ester compound (Compound 128) (5 g, 11 mmol), and tetrakistriphenylphosphinepalladium (Pd(PPh₃)₄) (1.2 g, 1.1 mmol) was added thereto. The mixture was dissolved in toluene (100 mL). To the solution, added were Aliquat 336 (0.5 g, 1.1 mmol) and aqueous 2M calcium carbonate solution (30 mL). The resultant mixture was stirred at 130° C. under reflux for 4 hours. To the precipitate formed, excess amount of methanol was poured to form solid, which was then dissolved in chloroform (300 mL). After filtration, the organic solvent was removed. Recrystallization from tetrahydrofuran (200 mL) gave the target compound (129) (BATPN-1) (3.3 g, overall yield: 36%).

¹H NMR (200 MHz, CDCl₃) δ=7.22-7.32 (m, 16H), 7.48-7.54 (m. 10H), 7.67-7.73 (m, 6H), 7.85-7.88 (m, 4H), 8.04-8.09 (t, 2H), 8.52-8.88 (m, 4H), 8.74 (s, 2H)

MS/FAB: 884.34 (found), 885.10 (calculated)

### Preparation Example 5

### Preparation of Compound (132)

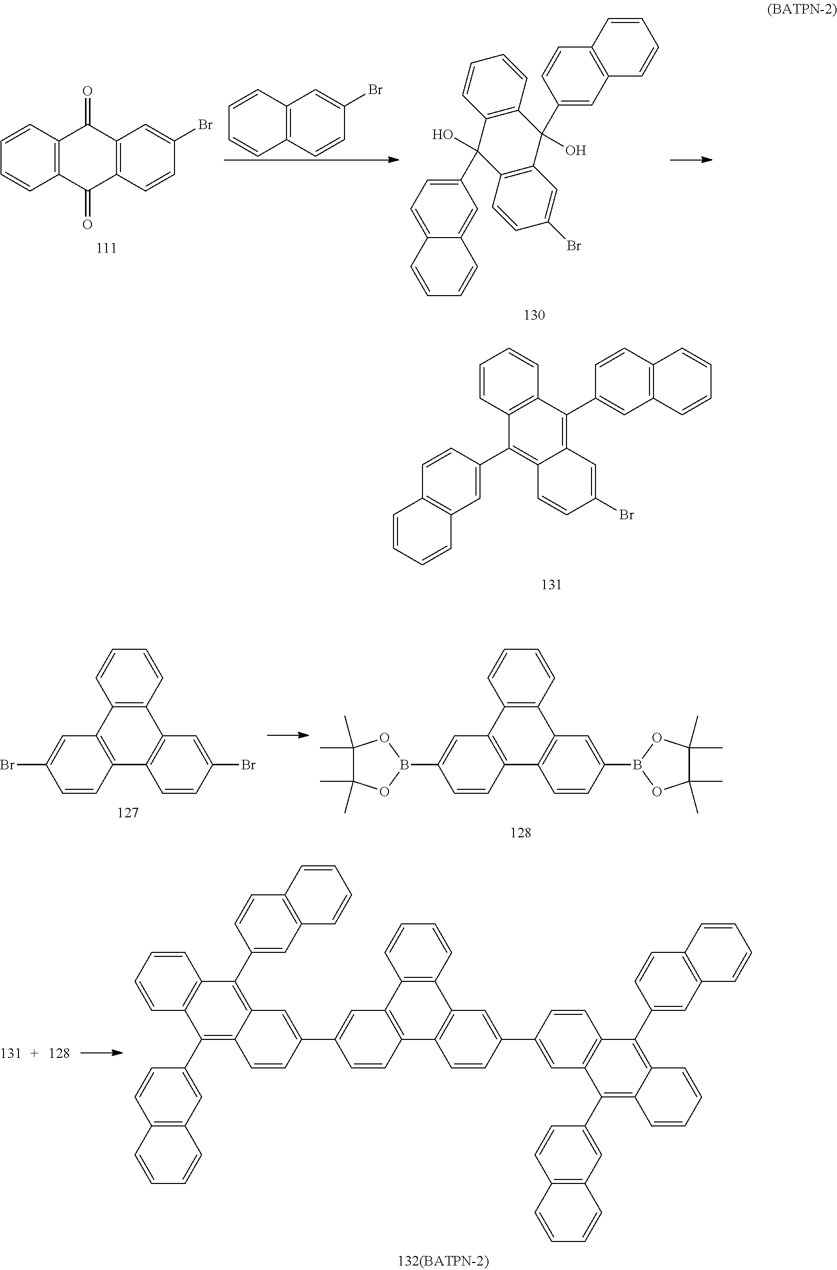

### Preparation of Compound (130)

Under nitrogen atmosphere, 2-bromonaphthalene (27.3 g, 132 mmol) was dissolved in tetrahydrofuran (250 mL), and n-BuLi (2.5 M in n-hexane) (79.2 mL, 198 mmol) was added dropwise at −78° C. thereto. After stirring for 2 hours, the reaction mixture was slowly added dropwise at −78° C. to a solution of 2-bromoanthraquinone (Compound III) (12.6 g, 44 mmol) dissolved in tetrahydrofuran (200 mL) at 25° C. under nitrogen atmosphere. The temperature was slowly raised from −78° C. to 25° C., and the reaction mixture was stirred for 12 hours. After quenching the reaction by adding saturated aqueous ammonium chloride solution (200 mL), the reaction mixture was extracted with ethyl acetate (200 mL) and dried over anhydrous magnesium sulfate. Evaporation of the organic layer under reduced pressure and recrystallization from dichloromethane (200 mL) gave Compound (130) (20.0 g, 36.8 mmol).

### Preparation of Compound (131)

A reaction vessel was charged with Compound (130) (19.2 g, 35.3 mmol), potassium iodide (23.4 g, 141.2 mmol) and sodium hydropotassiumphosphate hydrate (NaHPO₂H₂O) (22.5 g, 211.8 mmol). Glacial acetic acid (100 mL) was added thereto to dissolve the content of the vessel, and the solution was stirred under reflux. After 18 hours of stirring, the reaction mixture was cooled to 25° C., and distilled water was added thereto. The solid produced was filtered and washed with excess amount of water. Washing again with aqueous sodium hydroxide solution (300 mL) and recrystallization from hexane (200 mL) gave Compound (131) (16.0 g, 31.4 mmol).

### Preparation of Compound (128)

In tetrahydrofuran (120 mL), 1,8-dibromotriphenylene (Compound 127) (4.6 g, 12 mmol) was dissolved under nitrogen atmosphere, and n-BuLi (2.5 M in n-hexane) (14.4 mL, 35.9 mmol) was slowly added dropwise thereto at −78° C. After stirring for 1 hour, 2-isopropoxy-4,4,5,5-tetramethyl-1,3,2-dioxaborolane (23.4 g, 47.9 mmol) was added at low temperature, and the resultant mixture was stirred while slowly raising the temperature to 25° C. The reaction mixture was washed with water (500 mL), extracted with ethyl acetate (500 mL), and the extract was dried over anhydrous magnesium sulfate and filtered. Evaporation of the organic layer under reduced pressure and recrystallization from methanol (300 mL) gave solid, which was then filtered to obtain Compound (128) (5.0 g, 10.4 mmol).

### Preparation of Compound (132)

A reaction vessel was charged with Compound (131) (16 g, 30.9 mmol) and the ester compound (Compound 128) (5.0 g, 10.3 mmol), and tetrakistriphenylphosphinepalladium (Pd(PPh₃)₄) (1.2 g, 1.0 mmol) was added thereto. The mixture was dissolved in toluene (100 mL). To the solution, added were Aliquat 336 (0.5 g, 1.0 mmol) and aqueous 2M calcium carbonate solution (30 mL). The resultant mixture was stirred under reflux for 5 hours. To the precipitate formed, excess amount (300 mL) of methanol was poured to form solid, which was then dissolved in chloroform (500 mL). After filtration, the organic solvent was removed under reduced pressure. Recrystallization from tetrahydrofuran (200 mL) gave the target compound (132) (TPN-3) (3.6 g, overall yield: 38.9%).

¹H NMR (200 MHz, CDCl₃) δ=7.30-7.32 (m, 12H), 7.54-7.70 (m, 24H), 7.85-7.89 (m, 8H), 8.04-8.08 (t, 2H), 8.54-8.70 (m, 6H)

MS/FAB: 1084.41 (found), 1085.33 (calculated)

### Preparation Example 6

### Preparation of Compound (135)

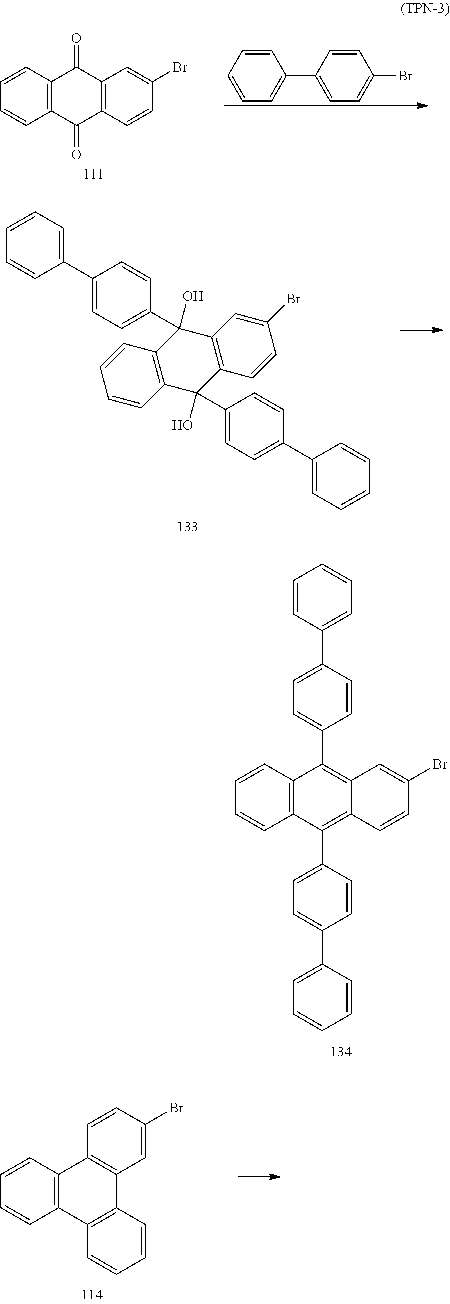

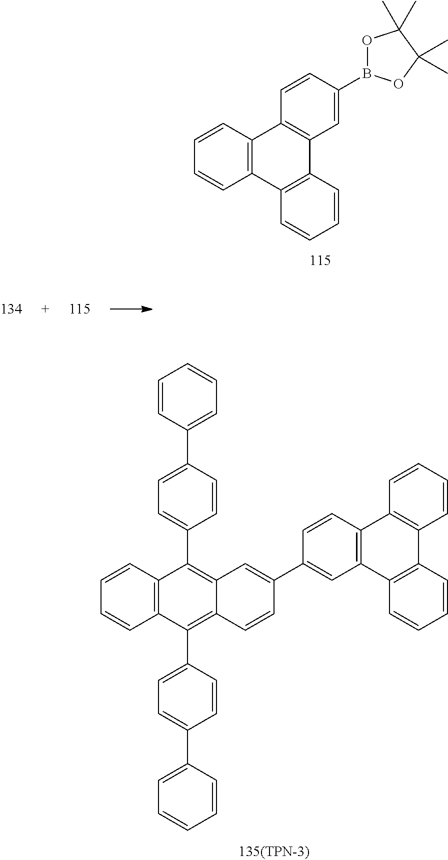

### Preparation of Compound (133)

Under nitrogen atmosphere, 4-bromobiphenyl (21 g, 90 mmol) was dissolved in tetrahydrofuran (200 mL), and n-BuLi (2.5 M in n-hexane) (54 mL, 135 mmol) was slowly added dropwise thereto at −78° C. After stirring for 2 hours, the reaction mixture was slowly added dropwise at −78° C. to a solution of 2-bromoanthraquinone (Compound III) (8.7 g, 30 mmol) dissolved in tetrahydrofuran (100 mL) at 25° C. under nitrogen atmosphere. The temperature was slowly raised from −78° C. to 25° C., and the reaction mixture was stirred for 12 hours. After quenching the reaction by adding saturated aqueous ammonium chloride solution (300 mL), the reaction mixture was extracted with ethyl acetate (300 mL), dried over anhydrous magnesium sulfate. Evaporation of the organic layer under reduced pressure and recrystallization from dichloromethane (200 mL) gave Compound (133) (14.8 g, 24.9 mmol).

### Preparation of Compound (134)

A reaction vessel was charged with Compound (133) (10 g, 16.8 mmol), potassium iodide (11.16 g, 67.2 mmol) and sodium hydropotassiumphosphate hydrate (NaHPO₂H₂O) (10.7 g, 100.8 mmol). Glacial acetic acid (100 mL) was added thereto to dissolve the content of the vessel, and the solution was stirred under reflux. After 18 hours of stirring, the reaction mixture was cooled to 25° C., and distilled water was added thereto. The solid produced was filtered and washed with excess amount of water. Washing again with aqueous sodium hydroxide solution (300 mL) and recrystallization from n-hexane (20 mL) gave Compound (134) (8.5 g, 15.5 mmol).

### Preparation of Compound (115)

In tetrahydrofuran (160 mL), 1-bromotriphenylene (Compound 114) (5.0 g, 16.2 mmol) was dissolved under nitrogen atmosphere, and n-BuLi (2.5 M in n-hexane) (9.7 mL, 21.0 mmol) was slowly added dropwise thereto at −78° C. After stirring for 1 hour, 2-isopropoxy-4,4,5,5-tetramethyl-1,3,2-dioxaborolane (6.0 g, 32.4 mmol) was added at low temperature, and the resultant mixture was stirred while slowly raising the temperature to 25° C. The reaction mixture was extracted with ethyl acetate (300 mL), and the extract was washed with water (300 mL), dried over anhydrous magnesium sulfate, and filtered. Evaporation of the organic layer under reduced pressure and recrystallization from methanol (200 mL) gave solid, which was then filtered and dried to obtain Compound (115) (5.0 g, 14.1 mmol).

### Preparation of Compound (135)

A reaction vessel was charged with Compound (134) (5.0 g, 8.9 mmol) and the ester compound (Compound 115) (4.7 g, 13.4 mmol), and tetrakistriphenylphosphinepalladium (Pd(PPh₃)₄) (1.0 g, 0.9 mmol) was added thereto. The mixture was dissolved in toluene (80 mL). To the solution, added were Aliquat 336 (0.4 g, 0.9 mmol) and aqueous 2M calcium carbonate solution (24 mL). The resultant mixture was stirred at 130° C. under reflux for 3 hours. To the precipitate formed, excess amount (200 mL) of methanol was poured to form solid, which was then dissolved in chloroform (500 mL). After filtration, the organic solvent was removed under reduced pressure. Recrystallization from tetrahydrofuran (200 mL) gave the target compound (135) (TPN-3) (2.0 g, overall yield: 32%).

¹H NMR (200 MHz, CDCl₃) δ=7.20-7.22 (m, 2H), 7.29-7.32 (m. 6H), 7.48-7.54 (m, 13H), 7.55-7.58 (m, 3H), 7.82-7.88 (m, 5H), 8.10-8.12 (m, 3H), 8.34 (dd, 1H), 8.93-8.99 (m, 3H)

MS/FAB: 708.28 (found), 708.89 (calculated)

### Preparation Example 7

### Preparation of Compound (140)

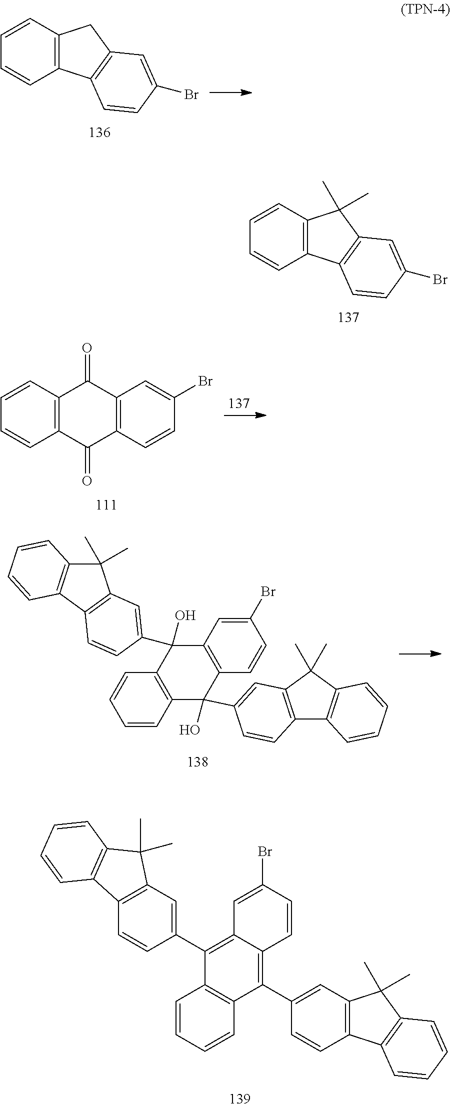

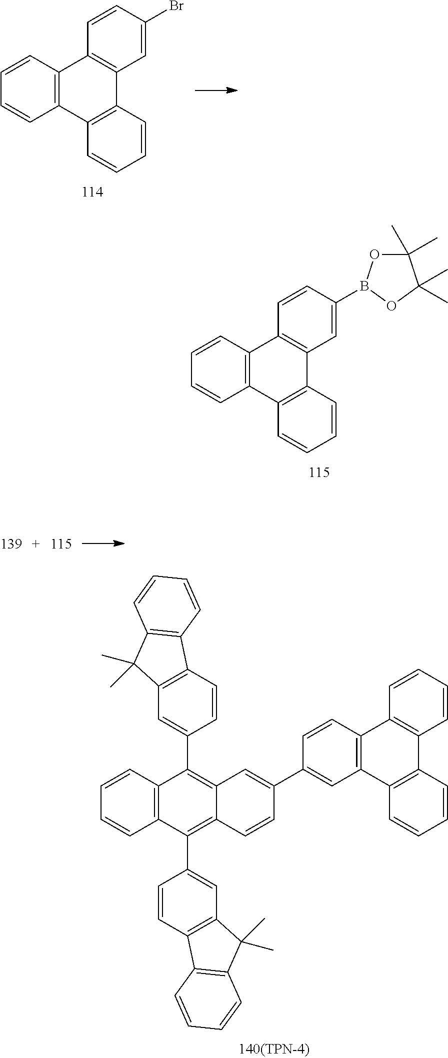

### Preparation of Compound (137)

Compound (136) (2-bromofluorene) (15.0 g, 60.0 mmol) and potassium hydroxide (26.7 g, 480.0 mmol) were added to methyl sulfoxide (300 mL), and the mixture was stirred. Distilled water (45 mL) was added thereto, and iodomethane (CH₃I) (33.9 g, 120 mmol) was added dropwise to the resultant mixture. After stirring for additional 20 minutes, the mixture was stirred at 25° C. for 20 hours. Water (500 mL) was added to quench the reaction. The mixture was extracted with dichloromethane (500 mL), and the organic layer was dried over anhydrous magnesium sulfate, and evaporated under reduced pressure. The compound obtained was purified by column chromatography (eluent: hexane), and dried to obtain Compound (137) (15.2 g, 55.6 mmol).

### Preparation of Compound (138)

Under nitrogen atmosphere, Compound (137) (14.3 g, 52.2 mmol) was dissolved in tetrahydrofuran (150 mL), and n-BuLi (2.5 M in n-hexane) (31.3 mL, 78.3 mmol) was slowly added dropwise at −78° C. thereto. After stirring for 2 hours, the reaction mixture was slowly added dropwise at −78° C. to a solution of 2-bromoanthraquinone (Compound III) (5.0 g, 17.4 mmol) dissolved in tetrahydrofuran (50 mL) at 25° C. under nitrogen atmosphere. The temperature was slowly raised from −78° C. to 25° C., and the reaction mixture was stirred for 12 hours. After quenching the reaction by adding saturated aqueous ammonium chloride solution (200 mL), the reaction mixture was extracted with ethyl acetate (200 mL), and the extract was dried over anhydrous magnesium sulfate and filtered. Evaporation of the organic layer under reduced pressure and recrystallization from hexane (200 mL) gave Compound (138) (9.9 g, 14.6 mmol).

### Preparation of Compound (139)

A reaction vessel was charged with Compound (138) (9.9 g, 14.6 mmol), potassium iodide (9.7 g, 58.4 mmol) and sodium hydropotassiumphosphate hydrate (NaHPO₂H₂O) (9.3 g, 87.7 mmol). Glacial acetic acid (30 mL) was added thereto to dissolve the content of the vessel, and the solution was stirred under reflux. After 18 hours of stirring, the reaction mixture was cooled to 25° C., and distilled water was added thereto. The solid produced was filtered and washed with excess amount of water. Washing again with aqueous sodium hydroxide solution (200 mL) and recrystallization from dichloromethane (200 mL) and hexane (200 mL) gave Compound (139) (8.5 g, 13.3 mmol).

### Preparation of Compound (115)

In tetrahydrofuran (200 mL), 1-bromotriphenylene

(Compound 114) (7.0 g, 22.8 mmol) was dissolved under nitrogen atmosphere, and n-BuLi (2.5 M in n-hexane) (13.6 mL, 34.1 mmol) was slowly added dropwise thereto at −78° C. After stirring for 1 hour, 2-isopropoxy-4,4,5,5-tetramethyl-1,3,2-dioxaborolane (8.5 g, 45.5 mmol) was added at low temperature, and the resultant mixture was stirred while slowly raising the temperature to 25° C. The reaction mixture was washed with water (300 mL), extracted with ethyl acetate (300 mL), and the extract was dried over anhydrous magnesium sulfate, and filtered. Evaporation of the organic layer under reduced pressure and recrystallization from methanol (200 mL) gave solid, which was then filtered to obtain Compound (115) (7.0 g, 19.9 mmol).

### Preparation of Compound (140)

A reaction vessel was charged with Compound (139) (8.5 g, 13.3 mmol) and the ester compound (Compound 115) (7.0 g, 19.9 mmol), and tetrakistriphenylphosphinepalladium (Pd(PPh₃)₄) (1.5 g, 1.3 mmol) was added thereto. The mixture was dissolved in toluene (150 mL). To the solution, added were Aliquat 336 (0.3 g, 1.3 mmol) and aqueous 2M calcium carbonate solution (45 mL). The resultant mixture was stirred at 130° C. under reflux for 5 hours. To the precipitate formed, excess amount of methanol was poured to form solid, which was then dissolved in chloroform (500 mL). After filtration, the solvent was removed under reduced pressure. Recrystallization from tetrahydrofuran (200 mL) gave the target compound (140) (TPN-4) (4.1 g, overall yield: 39%).

¹H NMR (200 MHz, CDCl₃) δ=1.67 (s, 12H), 7.28-7.36 (m, 6H), 7.84 (m, 19H), 8.10-8.12 (m, 3H), 8.34-8.36 (m, 1H), 8.93-8.99 (m, 3H)

MS/FAB: 788.34 (found), 789.01 (calculated)

### Preparation Example 8

### Preparation of Compound

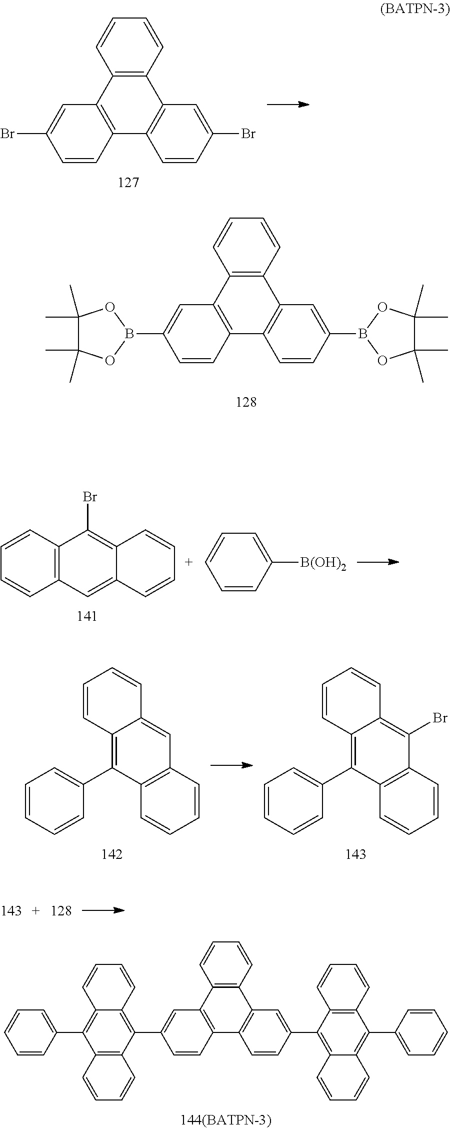

### Preparation of Compound (142)

Compound (141) (9-bromoanthracene) (15.0 g, 58.3 mmol), phenylboronic acid (8.5 g, 70.00 mmol) and tetrakistriphenylphosphinepalladium (Pd(PPh₃)₄) (6.7 g, 5.8 mmol) were dissolved in toluene (300 mL) and ethanol (150 mL). After adding aqueous 2 M sodium carbonate solution (486 mL), the mixture was stirred at 120° C. under reflux for 5 hours. The reaction mixture was then cooled to 25° C., and the reaction was quenched by adding distilled water (500 mL). The mixture was extracted with ethyl acetate (500 mL), and the organic layer was dried over anhydrous magnesium sulfate. Concentration of the organic layer under reduced pressure and recrystallization from tetrahydrofuran (200 mL) gave Compound (142) (12 g, 47.2 mmol).

### Preparation of Compound (143)

Compound (142) (11.7 g, 46.0 mmol) and N-bromosuccinimide (9.0 g, 50.6 mmol) were dissolved in dichloromethane (360 mL) under nitrogen atmosphere. The solution was stirred at 25° C. for 5 hours. After adding distilled water (400 mL) to quench the reaction, the reaction mixture was extracted with dichloromethane (400 mL). The organic layer obtained was dried over magnesium sulfate, filtered and concentrated under reduced pressure. Recrystallization from tetrahydrofuran (200 mL) gave Compound (143) (13.0 g, 39 mmol).

### Preparation of Compound (144)

A reaction vessel was charged with Compound (143) (10.4 g, 31.2 mmol) and the ester compound (Compound 128) (5.0 g, 10.4 mmol), and tetrakispalladiumtriphenylphosphine (Pd(PPh₃)₄) (1.2 g, 1.0 mmol) was added thereto. The mixture was dissolved in toluene (100 mL). To the solution, added were Aliquat 336 (0.5 g, 1.0 mmol) and aqueous 2M calcium carbonate solution (30 mL). The resultant mixture was stirred at 130° C. under reflux for 5 hours. To the precipitate formed, excess amount of methanol was poured to form solid, which was then dissolved in chloroform (300 mL). After filtration, the solvent was removed under reduced pressure. Recrystallization from tetrahydrofuran (200 mL) gave the target compound (144) (BATPN-3) (3.1 g, overall yield: 40%).

¹H NMR (200 MHz, CDCl₃) δ=7.20-7.32 (m, 14H), 7.48 (t. 4H), 7.67 (d, 8H), 7.84 (d, 2H), 8.04 (d, 2H), 8.49-8.55 (m, 4H), 8.70 (d, 2H)

MS/FAB: 732.28 (found), 732.91 (calculated)

### Preparation Example 9

### Preparation of Compound (147)

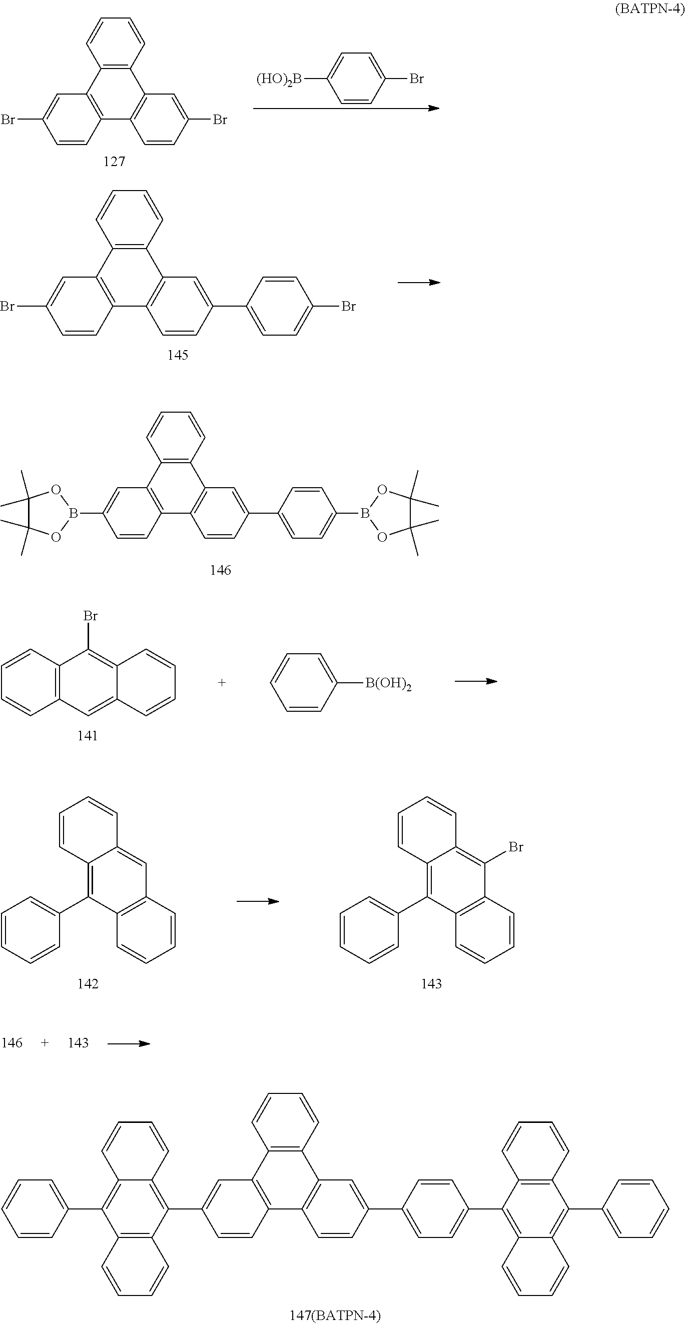

### Preparation of Compound (142)

Compound (141) (9-bromoanthracene) (15 g, 58.3 mmol), phenylboronic acid (8.5 g, 70.0 mmol) and tetrakistriphenylphosphinepalladium (Pd(PPh₃)₄) (6.7 g, 5.8 mmol) were dissolved in toluene (300 mL) and ethanol (150 mL). After adding aqueous 2 M sodium carbonate solution (486 mL), the mixture was stirred at 120° C. under reflux for 5 hours. The reaction mixture was then cooled to 25° C., and the reaction was quenched by adding distilled water (400 mL). The mixture was extracted with ethyl acetate (400 mL), and the organic layer was dried over anhydrous magnesium sulfate, and filtered. Concentration of the organic layer under reduced pressure and recrystallization from tetrahydrofuran (300 mL) gave Compound (142) (12.0 g, 47.2 mmol).

### Preparation of Compound (143)

Compound (142) (11.7 g, 46.0 mmol) and N-bromosuccinimide (9.0 g, 50.6 mmol) were dissolved in dichloromethane (360 mL) under nitrogen atmosphere. The solution was stirred at 25° C. for 5 hours. After adding distilled water (300 mL) to quench the reaction, the reaction mixture was extracted with dichloromethane (300 mL). The organic layer obtained was dried over magnesium sulfate, filtered and concentrated under reduced pressure. Recrystallization from tetrahydrofuran (200 mL) gave Compound (143) (13.0 g, 39.0 mmol).

### Preparation of Compound (145)

Compound (127) (1,8-dibromotriphenylene) (8.1 g, 20.8 mmol), 4-bromophenylboronic acid (4.6 g, 22.9 mmol) and trans-dichlorobistriphenylphosphine palladium (II) (1.5 g, 2.1 mmol) were dissolved in toluene (140 mL) and ethanol (70 mL). After adding 2 M sodium carbonate solution (100 mL) with stirring, the mixture was stirred with heating at 90° C. and reacted at the same temperature for 3 hours. The reaction mixture was then extracted with dichloromethane (300 mL), and the extract was washed with aqueous sodium chloride solution (300 mL) and filtered. Recrystallization from tetrahydrofuran (200 mL) gave Compound (145) (5.0 g, 10.8 mmol).

### Preparation of Compound (146)

In tetrahydrofuran (100 mL), Compound (145) (4.8 g, 10.3 mmol) was dissolved under nitrogen atmosphere, and n-BuLi (2.5 M in n-hexane) (12.4 mL, 31 mmol) was slowly added dropwise thereto at −78° C. After stirring for 1 hour, 2-isopropoxy-4,4,5,5-tetramethyl-1,3,2-dioxaborolane (7.7 g, 41.3 mmol) was added at low temperature, and the resultant mixture was stirred while slowly raising the temperature to 25° C. The reaction mixture was extracted with ethyl acetate (300 mL), washed with water (300 mL), dried over anhydrous magnesium sulfate, and filtered. Recrystallization from methanol (200 mL) gave solid, which was then filtered and dried to obtain Compound (146) (5.0 g, 9 mmol).

### Preparation of Compound (147)

A reaction vessel was charged with Compound (146) (9 g, mmol) and the ester compound (Compound 143) (5.0 g, 9 mmol), and tetrakistriphenylphosphinepalladium (Pd(PPh₃)₄) (1.0 g, 0.9 mmol) was added thereto. The mixture was dissolved in toluene (80 mL). To the solution, added were Aliquat 336 (0.4 g, 0.9 mmol) and aqueous 2M calcium carbonate solution (24 mL). The resultant mixture was stirred at 130° C. under reflux for 5 hours. To the precipitate formed, excess amount (300 mL) of methanol was poured to form solid, which was then dissolved in chloroform (500 mL). After filtration, the solvent was removed under reduced pressure. Recrystallization from tetrahydrofuran (200 mL) gave the target compound (147) (BATPN-4) (2.3 g, overall yield: 32%).

¹H NMR (200 MHz, CDCl₃) δ=7.22-7.32 (m, 14H), 7.48-7.54 (m. 8H), 7.67 (t, 8H), 7.82-7.88 (m, 2H), 8.04-8.18 (m, 4H), 8.34 (d, 1H), 8.93-8.99 (m, 2H), 9.15 (s, 1H)

MS/FAB: 809.32 (found), 809 (calculated)

### Example 1

### Manufacture of an OLED Using the Compound According to the Invention

An OLED was manufacture by employing an EL material according to the present invention.

First, a transparent electrode ITO thin film (15Ω/□) obtained from glass for OLED was subjected to ultrasonic washing with trichloroethylene, acetone, ethanol and distilled water, subsequently, and stored in isopronanol before use.

Then, an ITO substrate was equipped in a substrate folder of a vacuum vapor-deposit device, and 4,4′,4″-tris(N,N-(2-naphthyl)-phenylamino)triphenylamine (2-TNATA) was placed in a cell of the vacuum vapor-deposit device, which was then vented to reach 10⁻⁶ torr of vacuum in the chamber. Electric current was applied to the cell to evaporate 2-TNATA to vapor-deposit a hole injection layer with 60 nm of thickness on the ITO substrate.

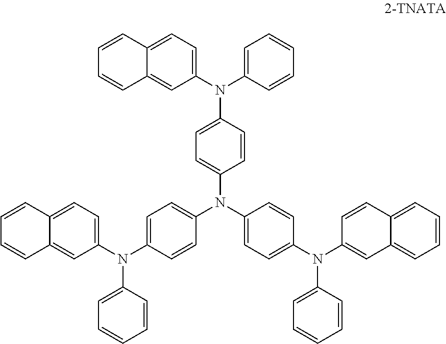

Then, another cell of the vacuum vapor-deposit device was charged with N,N′-bis(α-naphthyl)-N,N′-diphenyl-4,4′-diamine (NPB) (having the structural formula given below), and electric current was applied to the cell to evaporate NPB to vapor-deposit a hole transportation layer with 20 nm of thickness on the hole injection layer.

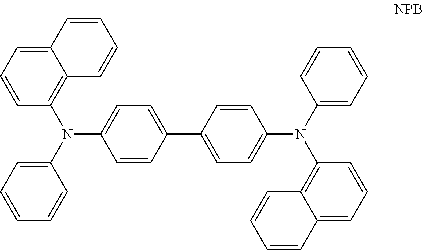

After formation of the hole injection layer and hole transportation layer, an EL layer was vapor-deposited as follows. One cell of the vacuum deposition device was charged with a compound according to the present invention (e.g. Compound TPN-4), while another cell of said device was charged with dopant EL materials having the structure shown below, respectively. With the vapor-deposition rate of 100:1, an EL layer was vapor-deposited with a thickness of 35 nm on the hole transportation layer.

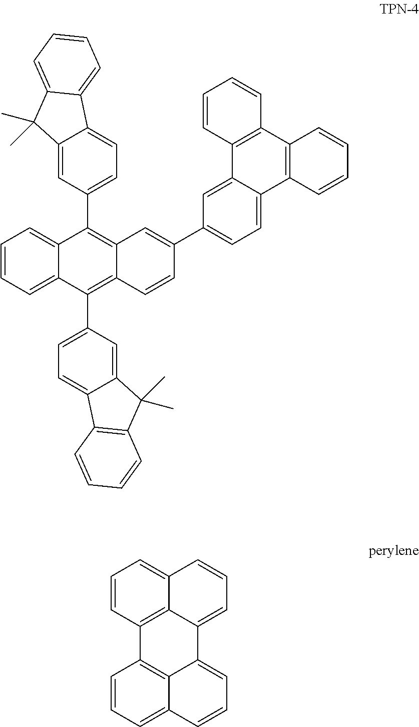

Then, tris(8-hydroxyquinoline)-aluminum (III) (Alq) was vapor-deposited with a thickness of 20 nm, as an electron transportation layer, followed by lithium quinolate (Liq) with a thickness of from 1 to 2 nm as an electron injection layer. Thereafter, an Al cathode was vapor-deposited with a thickness of 150 nm by using another vacuum vapor-deposit device to manufacture an OLED.

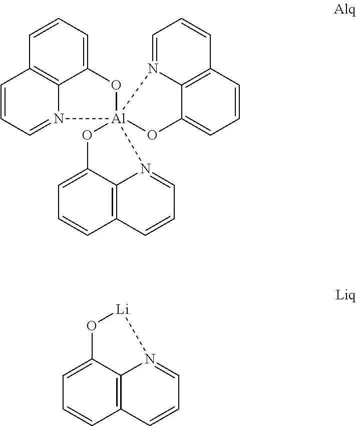

Individual EL materials used for the OLED devices were purified by vacuum sublimation under 10⁻⁶ torr.

### Comparative Example 1

### Manufacture of an OLED Using Conventional EL Material

A hole injection layer and hole transportation layer were formed according to the same procedure as described in Example 1, and dinaphthylanthracene (DNA) as a blue electroluminescent material was charged in one cell of said vapor-deposit device, while perylene in another cell as another blue electroluminescent material. Then, an electroluminescent layer with 35 nm thickness was vapor-deposited on said hole transportation layer with vapor-deposit rate of 100:1.

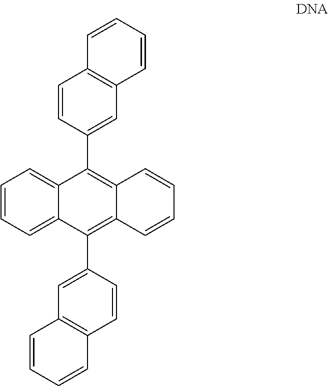

Then, an electron transportation layer and an electron injection layer were vapor-deposited according to the same procedure as described in Example 1, and an Al cathode was vapor-deposited by using another vacuum vapor-deposit device with a thickness of 150 nm, to manufacture an OLED.

### Example 2

### Electroluminescent Properties of the OLED Manufactured

Electroluminescent efficiencies of OLEDs comprising the organic electroluminescent compound according to the present invention prepared from Example 1 and the conventional electroluminescent compound from Comparative Example 1 were measured at 500 cd/m² and 2,000 cd/m², respectively, of which the results are shown in Table 1. Since the luminescent properties in the range of low luminance and those applied on the panel are very important in case of a blue electroluminescent material, in particular, the data of luminance of about 2,000 cd/m² was established as the standard in order to reflect those properties.

TABLE 1

| | | | | Luminous | | | | |
|---|---|---|---|---|---|---|---|---|
| | | | | efficiency | | Color | | |
| | EL | EL | EL peak | (cd/A) | | coordinate | | Luminous |
| No. | Material 1 | Material 2 | (nm) | @500 cd/m² | @2,000 cd/m² | X | Y | efficiency/Y |
| 1 | TPN-1 | Perylene | 460,488 | 5.22 | 4.96 | 0.160 | 0.212 | 23.4 |
| 2 | NTPN | Perylene | 466,494 | 6.10 | 5.37 | 0.163 | 0.230 | 23.3 |
| 3 | BPTPN | Perylene | 466,494 | 6.05 | 5.23 | 0.162 | 0.228 | 22.9 |
| 4 | BATPN-1 | Perylene | 470,498 | 6.62 | 5.93 | 0.165 | 0.250 | 23.7 |
| 5 | BATPN-2 | Perylene | 470,498 | 6.58 | 5.86 | 0.163 | 0.255 | 23.0 |
| 6 | TPN-3 | Perylene | 460,488 | 5.77 | 4.99 | 0.160 | 0.213 | 23.4 |
| 7 | TPN-4 | Perylene | 454,482 | 5.90 | 5.03 | 0.153 | 0.188 | 26.7 |
| 8 | BATPN-3 | Perylene | 454,482 | 5.88 | 4.90 | 0.155 | 0.195 | 25.1 |
| 9 | BATPN-4 | Perylene | 454,484 | 5.94 | 5.12 | 0.157 | 0.197 | 25.9 |
| Comp. 1 | DNA | Perylene | 456,484 | 4.45 | 3.62 | 0.160 | 0.200 | 22.3 |

As can be seen from Table 1, the OLED device employing the organic electroluminescent compounds according to the present invention as the electroluminescent material was compared to the OLED device of Comparative Example which employs widely known DNA:perylene as a conventional electroluminescent material, on the basis of “luminous efficiency/Y” value which shows similar tendency to quantum efficiency. As the result, the OLED device employing the organic electroluminescent compound according to the present invention showed higher “luminous efficiency/Y” value than that of Comparative Example.

Thus, the organic EL compounds according to the present invention can be employed as a high efficient blue EL material, including prominent advantages in terms of luminance and power consumption of an OLED as compared to conventional full-colored OLED's.

### INDUSTRIAL APPLICABILITY

The organic EL compounds according to the present invention provide good luminous efficiency and excellent life property, and thus enable to manufacture OLED devices with very good operation lifetime.

1. An organic electroluminescent compound represented by Chemical Formula (1): 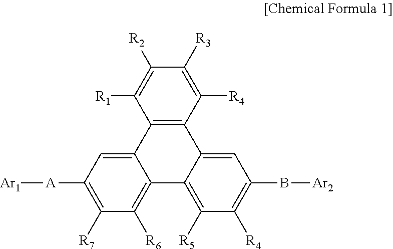
wherein, A and B independently represent a chemical bond or C₆-C₃₀ arylene;
R₁ through R₇ independently represent hydrogen, a C₁-C₂₀ linear or branched alkyl group, or a C₆-C₃₀ aryl group, or R₁ through R₇ may form a fused ring by an alkylene linkage with an adjacent group selected from R₁ to R₇;
Ar₁ and Ar₂ independently represent hydrogen, phenyl, naphthyl, anthryl or fluorenyl, wherein the phenyl, naphthyl, anthryl or fluorenyl may have one or more substituent(s) selected from the group consisting of C₁-C₂₀ linear or branched alkyl or alkoxy groups, C₆-C₃₀ aryl or heteroaryl groups and halogen; provided that Ar₁ and Ar₂ are not hydrogen at the same time; and
the arylene, aryl, heteroaryl, alkyl and alkoxy groups may be further substituted by C₁-C₂₀ linear or branched alkyl, aryl or halogen.

2. An organic electroluminescent compound according to claim 1, wherein A and B independently represent a chemical bond or a group represented by one of the following chemical formulas.

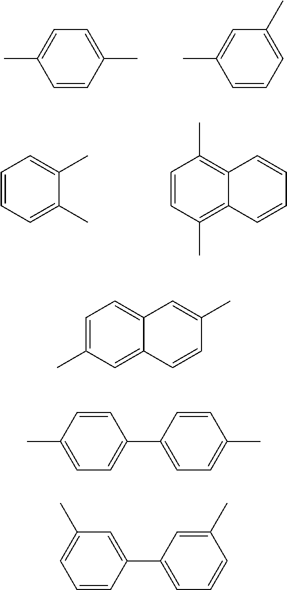

3. An organic electroluminescent compound according to claim 1, wherein group Ar₁ and Ar₂ independently represent an anthryl group having one of the following chemical formulas: 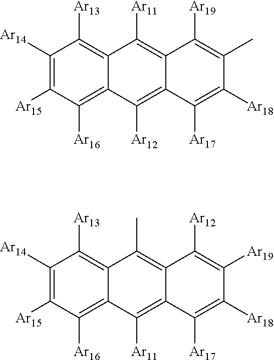
wherein, Ar₁ₗ through Ar₁₉ independently represent hydrogen, C₁-C₂₀ linear or branched alkyl or alkoxy group, C₆-C₃₀ aryl or heteroaryl group or halogen; and the aryl, heteroaryl, alkyl or alkoxy group may be further substituted by C₁-C₂₀ linear or branched alkyl, aryl or halogen.

4. An organic electroluminescent compound according to claim 1, wherein R₁ through R₇ may be independently selected from the group consisting of hydrogen, methyl, ethyl, propyl, isopropyl, butyl, isobutyl, pentyl, hexyl, ethylhexyl, heptyl, octyl, isooctyl, nonyl, decyl, dodecyl, hexadecyl, phenyl, tolyl, biphenyl, benzyl, naphthyl, anthryl and fluorenyl.

5. An organic electroluminescent compound according to claim 1, which is selected from the compounds represented by one of Chemical Formulas (2) to (5): 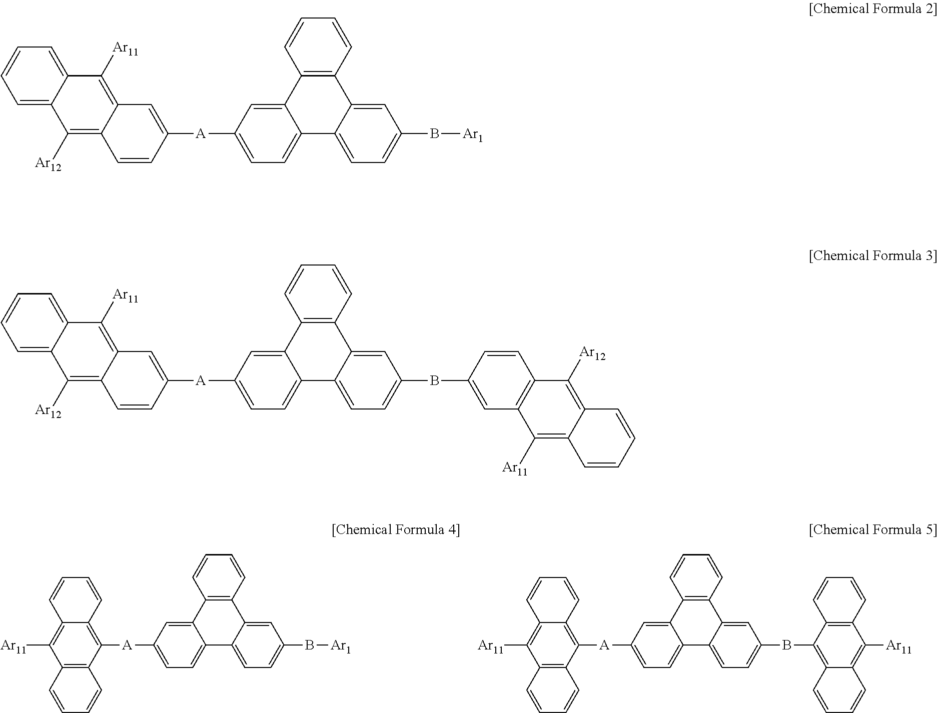
wherein, A and B are defined as in claim 1, and Ar₁, Ar₁ₗ and Ar₁₂ are independently selected from the group consisting of phenyl, 4-tolyl, 3-tolyl, 2-tolyl, 2-biphenyl, 3-biphenyl, 4-biphenyl, (3,5-diphenyl)phenyl, 9,9-dimethyl-fluoren-2-yl, 9,9-diphenyl-fluoren-2-yl, (9,9-(4-methylphenyl)-fluoren)-2-yl, 1-naphthyl, 2-naphthyl, 1-anthryl, 2-anthryl, 3-anthryl and 2-spirofluorenyl.

6. An organic electroluminescent compound according to claim 1, which is selected from the compounds represented by one of the following chemical formulas.

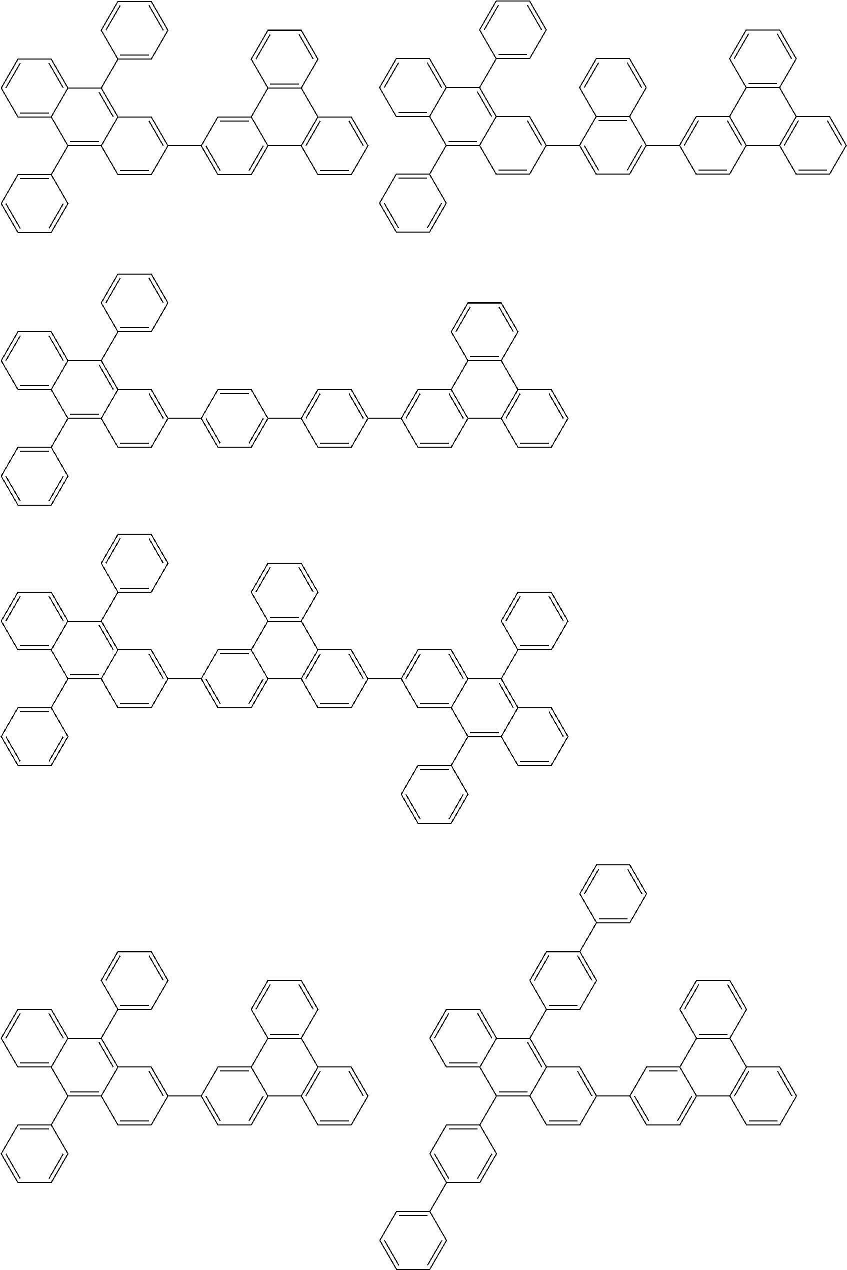

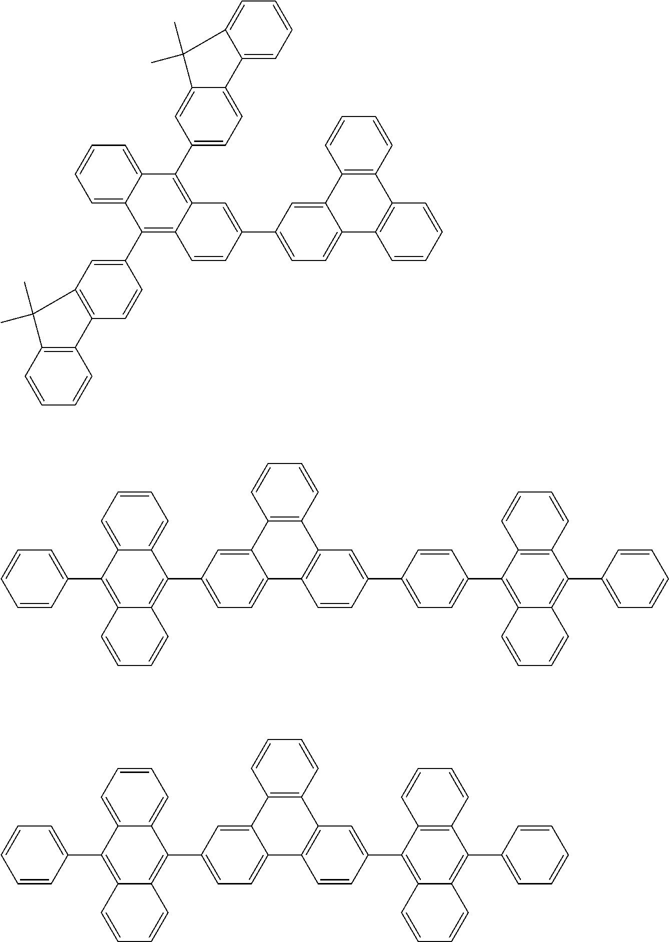

7. An organic electroluminescent device which comprises an organic electroluminescent compound according to any one of claims 1 to 6.
